(19) United States
(12) Patent Application Publication
(10) Pub. No.: US 2023/0173296 A1
(43) Pub. Date: Jun. 8, 2023

(54) INTRACAVITARY PHOTODYNAMIC THERAPY

(71) Applicant: Simphotek, Inc., Newark, NJ (US)

(72) Inventors: Mary POTASEK, Princeton, NJ (US); Karl BEESON, Princeton, NJ (US); Evgueni PARILOV, Brooklyn, NY (US)

(21) Appl. No.: 17/997,870

(22) PCT Filed: May 18, 2021
(86) PCT No.: PCT/US2021/032925

§ 371 Date: Nov. 3, 2022

Related U.S. Application Data
(60) Provisional application No. 62/704,602, filed on May 18, 2020.

(51) Int. Cl. A61N 5/06

(52) U.S. Cl. CPC A61N 5/0603; A61N 5/062; A61N 2005/0626; A61N 2005/063

(57) ABSTRACT

A method of generating a treatment plan for delivering treatment light for intracavitary photodynamic therapy to a targeted region within a cavity of a patient may include receiving shape information for an interior surface of the cavity. A first set of control points located on a first trajectory within the cavity is initialized by assigning, to each of the first set of control points, one or more axis positions of a treatment light emitter relative to the interior surface of the cavity. A simulated total treatment dose is iteratively optimized relative to a set of one or more optimization goals when the treatment light emitter is activated to emit treatment light at each of the first set of control points. The treatment plan is then generated to provide a total treatment dose to the targeted region.

### CROSS-REFERENCE TO RELATED APPLICATIONS

This application claims the benefit of priority to U.S. Provisional Application No. 62/704,602, filed on May 18, 2020, the entirety of which is incorporated herein by reference for all purposes.

### STATEMENT REGARDING FEDERALLY SPONSORED RESEARCH

This invention was made with government support under R01 grant number R01EB028778 awarded by the National Institute of Biomedical Imaging and Bioengineering of the National Institutes of Health. The government has certain rights in the invention.

### FIELD OF THE DISCLOSURE

The present disclosure relates generally to the field of intracavitary photodynamic therapy and related pretreatment planning methods and treatment delivery systems.

### BACKGROUND

Photodynamic therapy (PDT) is a treatment to kill cancerous cells, diseased cells or harmful bacteria that involves therapeutic photo-chemical interaction of light, photosensitizer (PS) and oxygen within tumor cells, diseased cells or harmful bacteria (See e.g. MacDonald et. al., “Basic principles of photodynamic therapy,” J. Nat. Cancer Inst. (1998) 90, 889-905; and Wilson et. al. “The physics of photodynamic therapy,” Phys. Med. Biol. (1986) 31, 327-360.). In PDT, a PS is injected into the body or a PS prodrug is applied superficially and may accumulate at higher PS concentrations in diseased tissue compared to normal tissue. Ideally PDT will generate the reactive species only within the target volume, leading to damage of the tumor or diseased tissues, while minimizing damage to surrounding normal tissue. Unlike chemotherapy, PDT does not cause systemic toxicities, and unlike radiation therapy it does not cause cumulative damage in the local treatment area. It should be noted that PDT is used in many applications in addition to cancer, such as oral cavity disease, blood products purification, cardiovascular diseases, autoimmune diseases, bacterial or viral infections, eye diseases and skin diseases.

The numerous PDT applications are characterized by multiple geometries. Most can be classified as consisting of surface (superficial), intracavitary or interstitial illumination. The use of surface illumination for PDT includes, but is not limited to, skin cancer and bacterial infections. The use of intracavitary illumination of the interior surface of a cavity for PDT includes, but is not limited to, applications such as oral cavity disease or cancer, disease or cancer of the gastrointestinal or respiratory tracts, thoracic cavity malignancies, bladder cancer and other cancers such as brain cancers where resection (removal) of primary tumors leaves a cavity. The use of interstitial illumination using optical fibers for PDT includes prostate and head and neck cancers, among others. The ability of PDT to spare surrounding critical normal structures and better healing after treatment distinguish the benefit of PDT compared to other localized therapeutic approaches, such as surgical resection (excision) or tissue X-ray radiation.

While PDT has demonstrated strong clinical efficacy data in some cases, PDT pretreatment planning and treatment delivery remain rudimentary for most clinical applications. Ideally, methods and systems for intracavitary PDT pretreatment planning and treatment delivery should be carried out via an established deterministic process which is based on the target region shape and properties and a sound computational model of the treatment. Conventional PDT pretreatment planning and treatment delivery that utilizes light dose (measured, for example, in J/cm²) as the dosimetry parameter neglects the concentration of the PS, the reaction kinetics of the PS and the quantitative formation of the reactive oxygen species, denoted as ROS. Singlet oxygen, ¹O₂, is the primary cytotoxic ROS that is responsible for cell death in Type II PDT, although other ROS can also be involved in Type I PDT. In particular, conventional PDT treatment planning and treatment delivery neglects the reactive oxygen species dose, [ROS]_{dose}, or concentration of reactive oxygen species produced. The [ROS]_{dose} depends on many parameters such as PS concentration, tissue optical properties, tissue oxygen concentration, oxygen intake rate from blood flow, light fluence rate (intensity or irradiance measured in mW/cm², for example) and treatment time. Inadequate pretreatment planning and treatment delivery can lead to increased rates of under- or over-treatment that manifest clinically as local recurrence or unnecessary local cell death.

There do not exist systems and methods for intracavitary PDT pretreatment planning and intracavitary PDT treatment delivery suitable for general patient PDT treatments. It would be desirable to develop methods for intracavitary PDT pretreatment planning and systems for intracavitary PDT treatment delivery that will be accurate for any cavity shape, any light transport parameters and any photokinetic parameters.

### SUMMARY

The embodiments disclosed herein are proposed to address the issues with PDT pretreatment planning and treatment delivery disclosed herein by optimizing pretreatment planning and treatment delivery for intracavitary PDT. The proposed new and improved pretreatment planning and treatment delivery methods and systems are accurate for any cavity shape, various light transport parameters and various photokinetic parameters. Both PDT-dose and [ROS]_{dose} depend on parameters such as PS photokinetic rate parameters, the initial PS concentration, PS photobleaching, tissue optical properties and changes in the tissue oxygen concentration during PDT. The embodiments of the present disclosure stem from the realization that prior art pretreatment planning and treatment delivery methods that calculate only light dose do not account for these parameters.

Some of the improved treatment planning methods and systems utilize, for example, Monte Carlo (MC) or Finite Element (FE) methods to initially calculate fluence rates for all portions of a target cavity, which is followed by photokinetic simulations to ensure that the entire target cavity receives at least a threshold PDT-dose or at least a threshold [ROS]_{dose}. It is also highly desirable to show treating physicians 3D visualizations of the resulting PDT-dose or [ROS]_{dose}, preferably superimposed on 3D images of the target cavity. In particular, it is important to show the PDT-dose or [ROS]_{dose} at the boundaries of the target cavity to ensure that all diseased or cancerous cells at the boundaries are treated with at least a threshold PDT-dose or a threshold [ROS]_{dose}.

Advantageously, the systems and methods described herein mitigate, alleviate or eliminate one or more deficiencies, disadvantages or issues in the art by providing optimization of pretreatment planning and treatment delivery for intracavitary photodynamic therapy.

Accordingly, in at least one embodiment, a method of delivering treatment light for intracavitary photodynamic therapy to a targeted region within a cavity of a patient may include generating a treatment plan for delivering the treatment light to generate a total treatment dose to the targeted region. The treatment plan is generated by receiving, at a processor, shape information for an interior surface of the cavity. A first set of control points located on a first trajectory within the cavity are initialized by the processor by assigning, to each of the first set of control points, one or more axis positions of a treatment light emitter relative to the interior surface of the cavity. The first trajectory defines a relative motion between a treatment light emitter and the interior surface of the cavity. A simulated total treatment dose relative to a set of one or more optimization goals is iteratively optimized by the processor when the treatment light emitter is activated to emit treatment light at each of the first set of control points. A treatment plan is determined by the processor by assigning values for a set of treatment light delivery parameters to each of the first set of control points. The treatment light is delivered to the targeted region within the cavity by activating the treatment light emitter in accordance with the treatment plan.

In accordance with at least one embodiment, a system for delivery of treatment light for intracavitary photodynamic therapy to a targeted region within a cavity of a patient may include a treatment light emitter, a positioning device and a controller. The positioning device is configured to move the treatment light emitter relative to a first set of control points located on a first trajectory within the cavity. The controller is configured to receive a treatment plan and, in accordance with the treatment plan, cause the positioning device to effect relative movement between the treatment light emitter and an interior surface of the cavity to enable the treatment light emitter to arrive at each of the first set of control points along the first trajectory. The controller is further configured to, while at each of the first set of control points, cause the treatment light emitter to be activated to emit light, and cause values of a set of treatment light delivery parameters of the treatment light emitter to vary in accordance with the treatment plan while the treatment light emitter is moved through the first set of control points along the first trajectory.

In accordance with at least one embodiment, a non-transitory computer-readable medium comprising instructions, which when executed by a processor, cause the processor to perform operations corresponding to any of the method described herein.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the present disclosure are described below with reference to the drawings. The illustrated embodiments are intended to illustrate, but not to limit, the present disclosure. The drawings contain the following figures:

FIG. 1 is a Jablonski diagram for formation of singlet oxygen and the reaction of singlet oxygen with target cells and with the photosensitizer.

FIG. 2 is a schematic illustration of a light delivery treatment planning system and a light delivery system for intracavitary PDT, in accordance with an embodiment of the present disclosure.

FIG. 3 is a schematic illustration of a portion of a light treatment delivery system for intracavitary PDT where light is emitted in all directions, in accordance with an embodiment of the present disclosure.

FIG. 4 illustrates a portion of a light treatment delivery system for intracavitary PDT where light is emitted only in some directions, in accordance with an embodiment of the present disclosure.

FIG. 5 illustrates a cross-sectional view of a cavity with a targeted region showing a trajectory with corresponding control points, in accordance with an embodiment of the present disclosure.

FIG. 6 illustrates a cross-sectional view of a cavity with a targeted region showing a trajectory with corresponding control points and light being delivered to the targeted region, in accordance with an embodiment of the present disclosure.

FIG. 7 illustrates a cross-sectional view of another cavity with a targeted region showing a trajectory with corresponding control points, in accordance with an embodiment of the present disclosure.

FIG. 8 illustrates a cross-sectional view of another cavity with a targeted region showing a trajectory with corresponding control points and light being delivered to the targeted region, in accordance with an embodiment of the present disclosure.

FIG. 9 illustrates a cross-sectional view of a cavity with a targeted region on only a portion of the cavity surface, showing a trajectory with corresponding control points, in accordance with an embodiment of the present disclosure.

FIG. 10 illustrates a cross-sectional view of a cavity with a targeted region on only a portion of the cavity surface, showing a trajectory with corresponding control points and light being delivered to the targeted region, in accordance with an embodiment of the present disclosure.

FIG. 11 is a flow chart illustrating a method for pretreatment planning utilizing a first trajectory, in accordance with an embodiment of the present disclosure.

FIG. 12 is a schematic illustration of a method for planning an delivery of light to a subject.

FIG. 13 is a flow chart illustrating a method for pretreatment planning utilizing a first trajectory and a second trajectory, in accordance with an embodiment of the present disclosure.

FIG. 14 is a schematic diagram of a treatment planning system, in accordance with an embodiment of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein.

### PDT Dosimetry Overview

PDT dosimetry involves determining the treatment dose delivered to cancerous, diseased or normal tissue at a fixed or variable dose rate, which is defined as the induced or delivered light fluence rate at each point in the target volume (i.e. irradiance in mW/cm²). There are three types of primary treatment dose metrics that are known to be used for PDT: (1) light dose (fluence) usually expressed in joules per centimeter squared (J/cm²), for example, which is equal the fluence rate (mW/cm² or mJ/(s cm²) times the time in seconds (s); (2) PDT-dose, usually expressed in μM J/cm², for example, which is defined as the time integral of the product of the local PS concentration times the fluence rate ϕ; and (3) reactive oxygen species dose, [ROS]_{dose}. For type II PDT, the reactive oxygen species dose, [ROS]_{dose} is equal to the reactive singlet oxygen dose, [¹O₂]_{dose}. For each type of dose, a threshold dose is needed to kill cancer cells.

### Light Dose

Standard PDT treatment planning of a treatment delivery protocol involves giving each patient the same total light dose (i.e. energy or incident fluence per area, J/cm²), determined for a particular type of cancer, irrespective of variations of PS concentration or other variables within a single target region or variations among different patients. An example of a light dose is, for example, 100 J/cm², although other higher or lower light doses have been previously utilized. Depending on other conditions such as PS concentration and oxygen intake rate, the conventional light dose may or may not result in an effective PDT-dose or [ROS]_{dose} or in an effective treatment. There is a threshold light dose needed to kill cells. This threshold can vary depending on treatment conditions such as the type of PS, the PS concentration, the initial oxygen concentration in the tissue, the oxygen flow rate delivered by blood flow to the tissue and the tissue optical properties. This variability in treatment conditions can lead to unreliable treatment results.

Some relevant PDT publications that include a threshold treatment light dose are: Sheng et. al. “Reactive oxygen species explicit dosimetry to predict local tumor control for Photofrin mediated photodynamic therapy,” Proc. SPIE 10860, 108600V (2019); Sheng et al., “Reactive oxygen species explicit dosimetry to predict tumor growth for benzoporphyrin derivative-mediated vascular photodynamic therapy”, J. Biomedical Optics 25(6), 063805 (2020); and Shafirstein et. al., “Irradiance controls photodynamic efficacy and tissue heating in experimental tumours: implication for interstitial PDT of locally advanced cancer”, British Journal of Cancer, 19, 1191-1199 (2018). Some examples of threshold light dose for animals are listed in TABLE 1. It is expected that a threshold light dose is also needed for treating human cancer tissue. See, for example, Davidson et al., “Treatment planning and dose analysis for interstitial photodynamic therapy of prostate cancer,” Phys. Med. Biol. 54 (2009) 2293-2313.

Without wishing to be bound by theory, the threshold doses may be reliable if the treated tissue has sufficient PS concentration and sufficient oxygen concentration to enable PDT. If the PS concentration or the oxygen concentration are too low, the target cancerous tissues may not be killed.

TABLE 1

| Photosensitizer | Animal | Threshold Light Dose [J/cm²] |
|---|---|---|
| Photofrin | Mice | Approx. 100 (Sheng et. al., 2019) |
| | Mice | ≥45 (Shafirstein et. al., 2018) |
| | Rabbits | ≥45 (Shafirstein et. al., 2018) |
| BPD | Mice | Approx. 40 (vascular, Sheng et. |
| | | al., 2020) |
| TOOKAD | Human prostate | >23 (Davidson et. al., 2009) |

### PDT-Dose

PDT-dose, usually expressed in μM J/cm², for example, is defined as the time integral of the product of the fluence rate ϕ times the local PS concentration. PDT-dose is usually more accurate for PDT treatment dosimetry than light dose because it takes into account the concentration of PS in the tissue, whereas light dose does not. For example, if the concentration of PS is very low or zero, no cancer will be killed even at a high light dose. Although PDT-dose will take the PS concentration into account, PDT-dose does not take into account the local oxygen concentration. If the oxygen concentration is too low, cancer cells will not be killed even for a high PDT-dose. There is a threshold PDT-dose needed to kill cells. This threshold can vary depending on conditions such as the type of PS, the PS concentration, the initial oxygen concentration in the tissue, the oxygen flow rate delivered by blood flow to the tissue and the tissue optical properties. Some relevant PDT publications that include a threshold PDT-dose are: Kim, et al; “Evaluation of singlet oxygen explicit dosimetry for predicting treatment outcomes of benzoporphyrin derivative monoacid ring A-mediated photodynamic therapy”; J Biomedical Optics, 22(2), 028002 (2017); Penjweini et al; “Evaluation of the 2-(1-Hexyloxyethyl)-2-devinyl pyropheophorbide (HPPH) mediated photodynamic therapy by macroscopic singlet oxygen modeling”; J. Biophotonics 9(11-12), 1344-1354 (2016); Sheng et. al. “Reactive oxygen species explicit dosimetry to predict local tumor control for Photofrin mediated photodynamic therapy,” Proc. SPIE 10860, 108600V (2019); Sheng, T et al; “Reactive oxygen species explicit dosimetry to predict tumor growth for benzoporphyrin derivative-mediated vascular photodynamic therapy”, J. Biomedical Optics 25(6), 063805 (2020).

Some PDT examples of threshold PDT-dose for animals are listed in TABLE 2. It is expected that a threshold PDT-dose is also needed for treating human cancer tissue. The threshold doses are only reliable if the treated tissue has sufficient oxygen concentration to enable PDT.

TABLE 2

| Photosensitizer | Animal | Threshold PDT-dose (μM J/cm²) |
|---|---|---|
| Photofrin | Mice | 439 (Sheng et. al., 2019) |
| BPD | Mice | 58 ± 12 (Kim et. al., 2017) |
| | | 7.5 (vascular, Sheng et. al., |
| | | 2020) |
| HPPH | Mice | 52.62 ± 14.9 (Penjweini et. al., |
| | | 2016) |

Reactive Oxygen Species Dose, [ROS]_{Dose}, or Reactive Singlet Oxygen Dose, [¹O₂]_{dose}

Recent experimental PDT work on tumors in mice indicates that the reactive oxygen species treatment dose, [ROS]_{dose}, or, in particular, the reactive singlet oxygen dose, [¹O₂]_{dose}, generated by the treatment light is more important than the conventional total light dose. See for example: Sheng, T et al; “Reactive oxygen species explicit dosimetry to predict tumor growth for benzoporphyrin derivative-mediated vascular photodynamic therapy”, J. Biomedical Optics 25(6), 063805 (2020); Penjweini, R. et al; “Evaluation of the 2-(1-Hexyloxyethyl)-2-devinyl pyropheophorbide (HPPH) mediated photodynamic therapy by macroscopic singlet oxygen modeling”; J. Biophotonics 9(11-12), 1344-1354 (2016) and Penjweini, R. et. al.; In-vivo outcome study of HPPH medicated PDT using singlet oxygen explicit dosimetry (SOED), Proc. of SPIE 2015; Vol. 9308, 93080N. The same light dose may result in differing amounts of generated singlet oxygen or other ROS depending on the treatment conditions such as the light fluence rate and oxygen intake from blood flow. It has been found in mice that a threshold [ROS]_{dose} should be reached during PDT treatment to successfully kill cancer cells. See, for example, Sheng et al, “Reactive oxygen species explicit dosimetry to predict tumor growth for benzoporphyrin derivative-mediated vascular photodynamic therapy”, J. Biomedical Optics 25(6), 063805 (2020). It is expected that a threshold [ROS]_{dose} or, in particular, a threshold [¹O₂]_{dose} will also be required to kill cancer cells in humans. This assumption cannot be directly tested since the experiments that were done on mice cannot be done on humans. The singlet oxygen threshold dose needed to kill cancer will depend on which PS is used for the treatment. Examples of threshold [¹O₂]_{dose} are shown in TABLE 3, where the data are taken from (a) Zhu et. al; “In-vivo singlet oxygen threshold doses for PDT”; Photon Lasers Med. 2015; 4(1), 59-71; (b) Qiu et al; “Macroscopic singlet oxygen modeling for dosimetry of Photofrin-mediated photodynamic therapy: an in-vivo study”; J. Biomedical Optics, 21(8), 088002 (2016); (c) Kim et al; “Evaluation of singlet oxygen explicit dosimetry for predicting treatment outcomes of benzoporphyrin derivative monoacid ring A-mediated photodynamic therapy”; J Biomedical Optics, 22(2), 028002 (2017); (d) Penjweini et al; “Evaluation of the 2-(1-Hexyloxyethyl)-2-devinyl pyropheophorbide (HPPH) mediated photodynamic therapy by macroscopic singlet oxygen modeling”; J. Biophotonics 9(11-12), 1344-1354 (2016). Note that the values of [¹O₂]_{Tdose} in TABLE 3 are labeled [¹O₂]ᵣₓ in the publications.

TABLE 3

| Photosensitizer | Animal | Threshold [¹O₂]_{dose} (mM) |
|---|---|---|
| Photofrin | Mice | 0.56 ± 0.26 (Zhu et. al., 2015) |
| | | >1.0 (Qiu et. al., 2016) |
| BPD | Mice | 0.72 ± 0.21 (Zhu et. al., 2015) |
| | | 0.98 ± 012 (Kim et. al. 2017) |
| HPPH | Mice | 0.98 ± 0.11 (Penjweini et. al., |
| | | 2016) |

### Light Dose Rate as a Secondary Dose Metric

In the case of using light dose as the primary metric, it has been found that a secondary dose metric may be useful. It was found that a threshold light dose rate ϕ_{T} (i.e. threshold irradiance in, for example, mW/cm²) is necessary to kill cancer in mice and rabbits. Shafirstein et. al., “Irradiance controls photodynamic efficacy and tissue heating in experimental tumours: implication for interstitial PDT of locally advanced cancer”, British Journal of Cancer, 19, 1191-1199 (2018). In particular, healing threshold light dose rates (see TABLE 4) have been shown for I-PDT treatment of mice and rabbits using the light dose as the dose metric.

TABLE 4

| | | Threshold Light Dose Rate ϕ_{T} |
|---|---|---|
| Photosensitizer | Animal | [mW/cm²] |
| Photofrin | Mice | 8.4 (Shafirstein et. al., 2018) |
| | Rabbits | 16.5 (Shafirstein et. al., 2018) |

### PDT Treatment Planning

In order to do accurate dosimetry in pretreatment planning and treatment delivery, computer simulations are performed. For intracavitary PDT, there exist prior art computer devices and software that can calculate PDT-dose, [¹O₂]_{dose} and PDT treatment dose rates, but only for a single fixed treatment light source position.

Input parameters include the treatment light source fluence rate ϕ, the target shape, the light source position relative to target shape, the optical parameters of the target and the PS concentration. Prior art computer devices and software for PDT are described in Beeson et. al., “Validation of combined Monte Carlo and photokinetic simulations for the outcome correlation analysis of benzoporphyrin derivative-mediated photodynamic therapy on mice,” J. Biomed. Opt 24(3), 035006, (2019a), and Beeson et. al. “Validation of Dosie™ combined Monte Carlo and photokinetic simulations for the analysis of HPPH-mediated photodynamic therapy on mice,” Proc. SPIE 10860, 108600N (2019b). These publications describe Monte Carlo simulations to determine light dose and treatment dose rates and photokinetic simulations that use the dose rate simulations to determine PDT-dose and [¹O₂]_{dose}. An example of photokinetic simulations to determine [¹O₂]_{dose} follows.

The amount of singlet oxygen [¹O₂] generated during PDT depends on the PS concentration and other parameters such as the amount of oxygen in the affected tissue, the amount of new oxygen that is being supplied to the tissue by blood vessels—the oxygen intake rate—the light fluence rate (mW/cm²) at the location of the PDT treatment and the treatment time. Note that a given threshold amount of singlet oxygen to kill cancer cells can be generated by a wide range of light fluence rates and treatment times.

In order to calculate the amount of singlet oxygen generated by intracavitary PDT at any point within cancerous tissue or on its surface, one must consider two separate calculations: (1) light transport through the tissue initiated by a given light fluence rate incident to the surface of the tumor tissue (also called direct light), where the transported light inside the tissue has a different light fluence rate after undergoing scattering and absorption by the tissue; and (2) the photokinetics of the modified light fluence rate interacting with the PS.

The light transport portion of the calculation can be addressed by, for example, Monte Carlo or finite element (FE) or by approximate solutions to the diffusion equation. Monte Carlo (MC) methods are a set of statistics based computational algorithms particularly suitable for simulations of complex systems. Distinct from most model-based techniques which produce solutions by solving a set of differential equations, Monte Carlo methods generate solutions by estimating the probability distribution after launching a large number of independent random trials. (See, for example, Fang et. al., “Monte Carlo simulation of photon migration in 3D turbid media accelerated by graphics processing units,” Optics Express 17(22), 20178 (2009), Beeson et. al., “Validation of combined Monte Carlo and photokinetic simulations for the outcome correlation analysis of benzoporphyrin derivative-mediated photodynamic therapy on mice,” J. Biomed. Opt 24(3), 035006 (2019a), and Beeson et. al. “Validation of Dosie™ combined Monte Carlo and photokinetic simulations for the analysis of HPPH-mediated photodynamic therapy on mice,” Proc. SPIE 10860, 108600N (2019b).) In contrast to MC methods, FE can be used to solve the time-dependent light diffusion approximation equation as a boundary value problem with appropriate initial conditions. See, for example, Oakley et al., “A New Finite Element Approach for Near Real-Time Simulation of Light Propagation in Locally Advanced Head and Neck Tumors Lasers,” Lasers in Surgery and Medicine 47, 60-67 (2015).

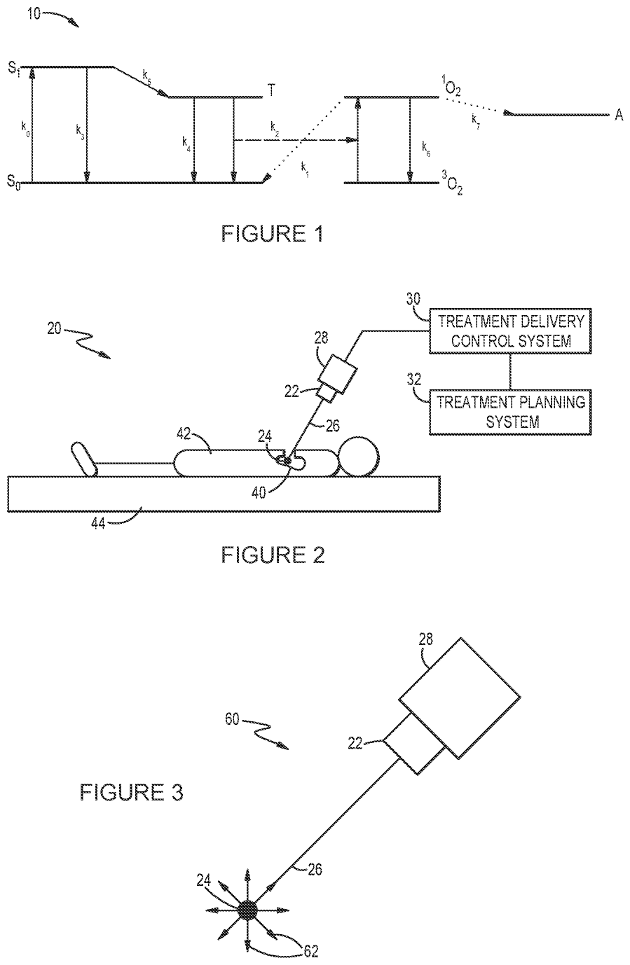

One can calculate the total amount of singlet oxygen generated by a PDT treatment by solving numerically the explicit kinetic rate equations of the PDT photo-chemical reactions. FIG. 1 shows Jablonski diagram 10 for single-photon photo-excitation of a photosensitizer (PS), resulting in the formation of singlet oxygen, ¹O₂, from ground state triplet oxygen, ³O₂. The singlet oxygen then can react and destroy cancer target cells, denoted as A, as well as react and destroy a portion of the PS ground state See e.g. Wang et. al; Explicit dosimetry for photodynamic therapy: macroscopic singlet oxygen modeling, J. Biophotonics 2010 June; 3(5-6); 304-318). The reactions can be described by the following set of coupled differential equations.

In the following equations, the concentration of the ground state of the PS is [S₀], the concentration of the first excited state of the PS is [S₁], the concentration of the triplet state of the PS is [T], the concentration of the ground state of oxygen is [³O₂], the concentration of the excited state of oxygen is [¹O₂], and the concentration of the cancer target is [A]. The photokinetic parameters k₀-k₇, g, δ, and S_{Δ} are defined in TABLE 5.

$$\frac{d [ {S}_{o} ]}{dt} = - {k}_{0} [ {S}_{o} ] - {k}_{1} [ {}^{1} {O}_{2} ] ( [ {S}_{0} ] + δ ) + {k}_{2} [ T ] [ {}^{3} {O}_{2} ] + {k}_{3} [ {S}_{1} ] + {k}_{4} [ T ] \qquad ( 1 )$$

$$\frac{d [ {S}_{1} ]}{dt} = ( {k}_{3} + {k}_{5} ) [ {S}_{1} ] + {k}_{0} [ {S}_{0} ] \qquad ( 2 )$$

$$\frac{d [ T ]}{dt} = - {k}_{2} [ T ] [ {}^{3} {O}_{2} ] + {k}_{4} [ T ] + {k}_{5} [ {S}_{1} ] \qquad ( 3 )$$

$$\frac{d [ {}^{3} {O}_{2} ]}{dt} = - {S}_{Δ} {k}_{2} [ T ] [ {}^{3} {O}_{2} ] + {k}_{6} [ [ {}^{1} {O}_{2} ] + g \qquad ( 4 )$$

$$\frac{d [ {}^{1} {O}_{2} ]}{dt} = - {k}_{1} ( [ {S}_{0} ] + δ ) [ {}^{1} {O}_{2} ] + {S}_{Δ} {k}_{2} [ T ] [ {}^{3} {O}_{2} ] - {k}_{6} [ {}^{1} {O}_{2} ] - {k}_{7} [ A ] [ {}^{1} {O}_{2} ] \qquad ( 5 )$$

$$\frac{d [ A ]}{dt} = - {k}_{7} [ A ] [ {}^{1} {O}_{2} ] \qquad ( 7 )$$

The system of differential equations (1)-(6) can be solved numerically (See e. g. Zhu et. al; Macroscopic modeling of the singlet oxygen production during PDT; Proc. SPIE 2007; Vol. 6427, 642708; and Potasek et. al.; Calculation of singlet oxygen formation from one photon absorbing photosensitizers used in PDT; Proc. SPIE 2013; Vol. 8568, 85681D). However, there are two issues here. First, the rate parameters, k₀-k₇, may not be accurately known for a photosensitizer in living tissue. Second, since the treatment time scales for the reactions in Equations (1)-(6) range from nanoseconds to thousands of seconds, numerical calculations can take hours to complete which is undesirable for treatment planning.

To get around these issues, approximate solutions to Equations (1)-(6) have been described. (See e.g. Wang et. al; Explicit dosimetry for photodynamic therapy: macroscopic singlet oxygen modeling, J. Biophotonics 2010 June; 3(5-6), 304-318) Since the lifetimes of [S₁], [T] and [¹O₂] are short compared to [S₀] and [³O₂], then [S₁], [T] and [¹O₂] are treated as reaching steady state relative to [S₀] and [³O₂]. The derivatives in equations (2), (3) and (5) are set equal to zero as shown in equations (7), (8) and (9).

$$\frac{d [ {S}_{1} ]}{dt} = 0 \qquad ( 7 )$$

$$\frac{d [ T ]}{dt} = 0 \qquad ( 8 )$$

$$\frac{d [ {}^{1} {O}_{2} ]}{dt} = 0 \qquad ( 9 )$$

As explained in e.g. Wang et. al; Explicit dosimetry for photodynamic therapy: macroscopic singlet oxygen modeling, J. Biophotonics 2010 June; 3(5-6), 304-318, the six Equations (1)-(6) then reduce to the following three Equations (10)-(12). One can solve the system of Equations (10)-(12) numerically and determine the total singlet oxygen dose, [¹O₂]_{dose}, which is denoted in Wang, K. K. et. al. publication as [¹O₂]ᵣₓ. Alternatively, one can solve for [ROS]_{dose} using the equivalent set of equations as described in Sheng et. al.; Reactive oxygen species explicit dosimetry to predict tumor growth for benzoporphyrin derivative-mediated vascular photodynamic therapy, J. Biomed. Opt. 2020; 25(6), 063805. For simplicity, it can be assumed that the constant S_{Δ}=0 and the constant δ=0.

$$\frac{d [ {S}_{0} ]}{dt} = - \frac{[ {}^{3} {O}_{2} ]}{[ {}^{3} {O}_{2} ] + β} ( [ {S}_{0} ] + δ ) ϕ [ {S}_{0} ] ξ σ \qquad ( 10 )$$

$$\frac{d [ {}^{3} {O}_{2} ]}{dt} = - \frac{[ {}^{3} {O}_{2} ]}{[ {}^{3} {O}_{2} ] + β} ϕ [ {S}_{0} ] ξ + g ( 1 - \frac{[ {}^{3} {O}_{2} ]}{{[ {}^{3} {O}_{2} ]}_{0}} ) \qquad ( 11 )$$

$$\frac{{d [ ROS ]}_{dose}}{dt} = ξ \frac{[ {}^{3} {O}_{2} ]}{[ {}^{3} {O}_{2} ] + β} ϕ [ {S}_{0} ] \qquad ( 12 )$$

The total singlet oxygen dose can be found by integrating Equation (12) from time t=0 to the treatment time, T, which gives:

$${[ ROS ]}_{dose} = {\int }_{0}^{T} ξ \frac{[ {}^{3} {O}_{2} ]}{[ {}^{3} {O}_{2} ] + β} ϕ [ {S}_{0} ] dt \qquad ( 13 )$$

The parameters used in Equations (10)-(13) are defined in TABLE 5. The ξ, σ and δ parameters are related to the rates k₁-k₇ and [A]. The ξ, σ and δ parameters can differ for different photosensitizers and can be determined experimentally. The light fluence rate is denoted as ϕ. Equations (10)-(13) can be (13) solved by standard numerical integration techniques (e.g., Runge-Kutta method) when the starting values of the PS concentration, [S₀ (t=0)], the tissue oxygen concentration, [³O₂ (t=0)], and the oxygen intake rate, g, are specified. Although Equation (13) can be solved for different fluence rates, it is not obvious how to determine an optimum range of fluence rates what can simultaneously minimize both the laser light power and minimize the therapeutic time to reach the threshold value of total singlet oxygen dose [¹O₂]_{Tdose}. A table of kinetic parameters for a variety of photosensitizers can be found in Kim et. al.; On the in vivo photochemical rate parameters for PDT reactive oxygen species modeling, Phys. Med. Biol. 62 (2017) R1-R48.

TABLE 5

| Parameter | Definition | Units |
|---|---|---|
| k₀ | Photon absorption rate of PS per PS concentration | s⁻¹ |
| k₁ | Bimolecular rate for ¹O₂ reaction with PS ground state S₀ | s⁻¹μM⁻¹ |
| k₂ | Bimolecular rate of PS triplet T state quenching by ³O₂ | s⁻¹μM⁻¹ |
| k₃ | Decay rate of PS first excited state S₁ | s⁻¹ |
| k₄ | Rate of decay of PS triplet state T | s⁻¹ |
| k₅ | Decay rate of PS first excited state S₁ to triplet state T | s⁻¹ |
| k₆ | ¹O₂ to ³O₂ decay rate | s⁻¹ |
| k₇ | Bimolecular rate of reaction of ¹O₂ with cancer target A | s⁻¹μM⁻¹ |
| S_{Δ} | Fraction of PS triplet state T to ³O₂ reactions to produce ¹O₂ | — |
| δ | Low concentration correction | μM |
| g | ³O₂ oxygen intake rate | μMs⁻¹ |
| ξ | ${S}_{Δ} ( \frac{{k}_{5}}{{k}_{5} + {k}_{3}} ) \frac{ε}{h v} ( \frac{{k}_{7} [ A ] / {k}_{6}}{{k}_{7} [ A ] / {k}_{6} + 1} )$ | cm²mW⁻¹s⁻¹ |
| σ | k₁/(k₇[A]) | μM⁻¹ |
| ε | PS extinction coefficient | cm⁻¹μM⁻¹ |
| hv | Energy of one photon; h is Planck’s constant; v is the | Joules |
| | photon frequency | |
| β | k₄ + k₂ | μM |
| ϕ | Light fluence rate | mW/cm² |
| [S₀ (t = 0)] | Initial PS concentration at time t = 0 | μM |
| [³O₂ (t = 0)] | Initial ground state oxygen concentration at time t = 0 | μM |

Some experimentally determined values for the ε, ξ and β parameters for different photosensitizers are listed in TABLE 6. (See e. g. Zhu et. al; In-vivo singlet oxygen threshold doses for PDT; Photon Lasers Med. 2015; 4(1), 59-71.)

TABLE 6

| Parameter (units) | Photosensitizer (wavelength) |
|---|---|
| ε (cm⁻¹ μM⁻¹) | Photofrin (630 nm): 0.0035 |
| | mTHPC (650 nm): 0.048 |
| | BPD (690 nm): 0.0783 |
| ξ (cm²mW⁻¹s⁻¹) | Photofrin: 3.7 × 10⁻³ |
| | mTHPC: 30.0 × 10⁻³ |
| | BPD: 51.0 × 10⁻³ |
| σ (μM⁻¹) | Photofrin: 7.6 × 10⁻⁵ |
| | mTHPC: 2.97 × 10⁻⁵ |
| | BPD: 1.7 × 10⁻⁵ |
| β (μM) | Photofrin: 11.9 |
| | mTHPC: 8.7 |
| | BPD: 11.9 |

The kinetic Equations (10)-(13) are also needed in order to calculate PDT-dose. To calculate PDT-dose, which is defined as the time integral of the product of the fluence rate ϕ times the local PS concentration, one can solve Equation (10) for the PS concentration (i.e. [S₀] in Equation (10)) as a function of time, multiply the PS concentration by the fluence rate ϕ and then calculate the time integral of the result.

Referring now to FIG. 1, which shows Jablonski diagram 10 for photo-excitation of an electron of a photosensitizer (PS) from a ground state S₀ to an excited state S₁ at rate k₀, transferring an electron at rate k₅ to a triplet state T which undergoes energy transfer from state T to oxygen at rate k₂ resulting in the formation of singlet oxygen, ¹O₂, from ground state triplet oxygen, ³O₂. The singlet oxygen then can react and destroy cancer target cells at rate k₇, denoted as A, as well as react and destroy at rate k₁ a portion of the PS ground state, which is labeled S₀.

The present disclosure, thus, provides a system for pretreatment planning and treatment delivery for intracavitary photodynamic therapy. FIG. 2 depicts an embodiment of a system 20 for treatment planning and light delivery for intracavitary PDT. The cavity 40 to be treated may be a natural cavity including, but not limited to, mouth, nasal cavity, trachea, bronchial tube, thoracic cavity, lung or bladder. Alternatively, the cavity 40 may result when a surgeon removes a substantial portion of a cancerous tumor or other tissue.

The treatment planning and light delivery system 20 comprises at least one light source 22, at least one treatment light emitter 24 that emits the PDT treatment light, at least one light transporting device 26 such as an optical fiber device for transporting treatment light from the light source 22 to the treatment light emitter 24, a multi-axis mechanical or robotic positioning device 28 to control the position of the treatment light emitter 24, a treatment delivery control system 30 and a treatment planning system 32.

In some embodiments, the light transporting means 26 is optional and may not be needed if the light source 22 is small and can be utilized directly as the treatment light emitter 24. Typically, however, the light source 22 is too large to be placed inside the cavity and a light transporting means 26 is needed.

The types of light source 22 may include, but are not limited to, one or more lasers or one or more light emitting diodes (LEDs). Lasers may include, but are not limited to, solid-state lasers, gas lasers, diode lasers, fiber lasers, continuous lasers and pulsed lasers. LEDs may include devices constructed from inorganic or organic materials (OLEDs). The lasers or LEDs emit light of the appropriate wavelength or wavelengths to be absorbed by the PS to generate singlet oxygen.

The treatment light emitter 24 may be a single emitter or a plurality of emitters. The treatment light emitter 24 may a diffusing emitter that emits light in all directions (for example, an isotropic or nearly isotropic beam that is directed in substantially all directions) or the treatment light emitter 24 may emit light in particular direction or directions (for example, a cone of light with a fixed or variable angular spread). An example of a diffusing treatment light emitter 24 is a cylindrical diffusing fiber. Cylindrical diffusing optical fibers can be are commercially available and may be obtained from vendors such as Medlight S.A., Switzerland.

The multi-axis mechanical positioning device 28 may be, for example, a three axis positioning device capable of moving the treatment light emitter 24 in three directions (x, y, z). In some embodiments, the mechanical positioning device 28 may be a six-axis positioning device capable of moving the treatment light emitter 24 in six directions (x, y, z, r, θ, φ). The directions r, θ, and φ, if used, adjust the angular orientation and radial extension of the light transporting means 26 and treatment light emitter 24. The multi-axis mechanical positioning system may sequentially position the treatment light emitter 24 by controlling one or more axis positions of the treatment light source relative to the cavity of the patient.

The light transporting device 26 may be a single optical fiber device or a plurality of optical fiber devices. If the light transporting device 26 is one or more glass or polymer optical fiber devices, each optical fiber device may include, but is not limited to, a single-mode optical fiber, a multimode optical fiber, a hollow core optical fiber, a photonic crystal fiber or a polarization-preserving fiber. The light transporting device 26 may also include free-space light propagation that may direct light to the treatment light emitter 24 with mirrors, lenses, prisms, beam-splitters, and/or other optical elements.

The treatment delivery control system 30 generally comprises hardware and/or software components. The treatment delivery control system 30 receives information from the treatment planning system 32 and controls the position of the treatment light emitter 24 along one or more trajectories as well as controlling the optical power of the treatment light emitter 24 and treatment light exposure times for the treatment light.

The treatment planning system 32 generally comprises computer hardware and/or software components and includes its own controller which is configured to execute suitable software. The computer hardware may comprise one or more central processing units (CPUs) and/or one or more process accelerating units. The process accelerating units may include, but are not limited to, graphical processing units (GPUs), field programmable units such as field-programmable gate arrays (FPGAs) and application specific integrated circuits (ASICs).

In some embodiments, inputs to the treatment planning system 32 may include, for example, the cavity shape, tissue optical properties, and, optionally, PS concentration, oxygen concentration and PS rate parameters. The cavity shape may be obtained by imaging technics such as, for example, computed tomography (CT), positron emission tomography (PET), magnetic resonance imaging (MM), ultrasound imaging or 3D optical imaging.

The details of the treatment planning system 32 are further described with reference to FIG. 14. In some embodiments, the treatment planning system 32 optimizes a treatment plan prior to treatment delivery in order to determine treatment parameters for the treatment light emitter 24 along one or more trajectories. The parameters may include, but are not limited to, the treatment light power (sometimes expressed in milliwatts or mW) from the treatment light emitter in order to achieve the desired intensity or fluence rate in milliwatts per square centimeter (mW/cm²) at the targeted region, the treatment light exposure times to reach the total treatment dose at the targeted region and, optionally, the treatment light emitted shape. The total treatment dose may be a light dose, preferably a PDT-dose, and more preferably a reactive oxygen species dose or a reactive singlet oxygen dose. When utilizing light dose, preferably the light dose is greater than a threshold light dose. When utilizing PDT-dose, preferably the PDT-dose is greater than a threshold PDT-dose. When utilizing reactive oxygen species dose, preferably the reactive oxygen species dose is greater than a threshold reactive oxygen species dose. When utilizing reactive singlet oxygen dose, preferably the reactive singlet oxygen dose is greater than a threshold singlet oxygen dose.

The treatment planning and light delivery system 20 controls motion of the treatment light emitter 24 to deliver treatment light to the targeted region of cavity 40 of patient 42. Patient 42 is positioned on platform support 44 which is typically stationary during treatment.

FIG. 3 is a schematic diagram of a portion of a light delivery system 60 for intracavitary PDT. The portion of the light delivery system 60 comprises a multi-axis mechanical or robotic positioning device 28 to control the position of the treatment light emitter 24 and optional light transporting device 26 such as an optical fiber device for transporting treatment light from the light source 22 to the treatment light emitter 24. In this example, light rays 62 are emitted in substantially all directions from the treatment light emitter 24.

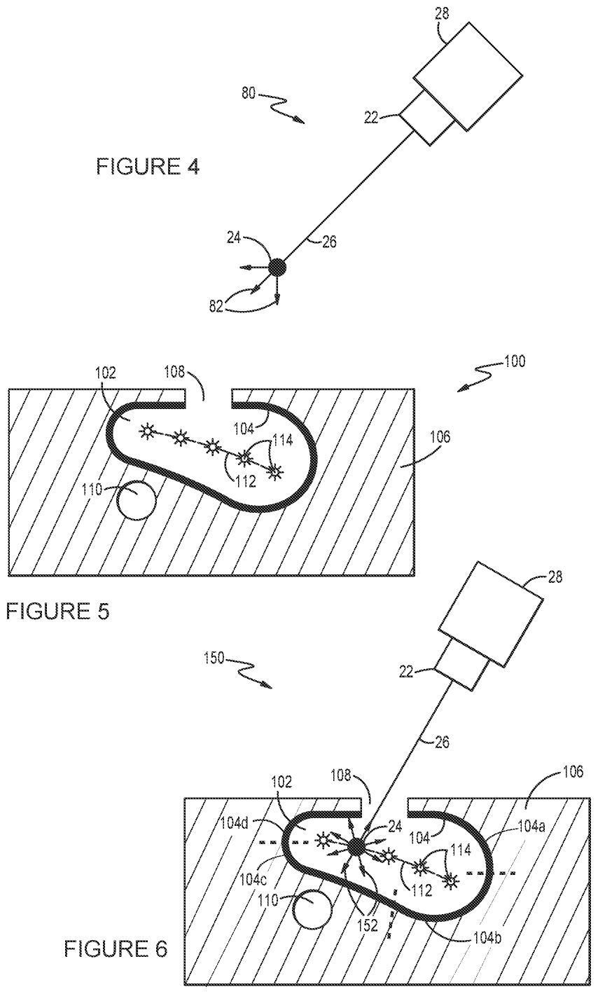

FIG. 4 is a schematic diagram of a portion of a light delivery system 80 for intracavitary PDT. The portion of the light delivery system 80 comprises a multi-axis mechanical or robotic positioning device 28 to control the position of the treatment light emitter 24 and a light transporting device 26 such as an optical fiber device for transporting treatment light from the light source 22 to the treatment light emitter 24. In this example, light rays 82 are emitted in a particular direction or directions (for example, the treatment light emitted shape is a cone of light with a fixed or variable angular spread) from the treatment light emitter 24.

FIG. 5 is an illustrative cross-sectional view of a portion 100 of a patient comprising a cavity 102 with a targeted region 104 that is highlighted as a black band and is located inside healthy tissue 106. The cavity has an opening 108 through which treatment light may enter. An organ-at-risk (OAR) 110 is located near the targeted region 104. The OAR 110 may be sensitive to the total treatment dose and should be subject to significantly less than the threshold value of the total treatment dose needed to treat the targeted region 104.

An example trajectory 112 for the path of the treatment light emitter 24 (shown in FIG. 2) is illustrated along with five control points 114. The trajectory 112 may be determined manually or by computer calculation. The trajectory 112 and control points 114 are optimized by the treatment planning system 32 (shown in FIG. 2). In this illustrative example, there are five control points where light is emitted by the treatment light emitter. Light may by emitted only at discrete control points 114 or light may be emitted continuously along the trajectory 112.

If light is emitted at discrete control points, for example, the treatment light power and treatment light exposure times may be different for each control point in order to deliver a uniform or nearly uniform threshold value of the total treatment dose to the entire targeted region 104 while sparing the OAR 110 from a damaging dose. If light is emitted continuously along the trajectory 112, the computer processor or processors in the treatment delivery control system 30 (shown in FIG. 2) need to be fast enough to continuously calculate the total treatment dose as the treatment light emitter moves along the trajectory 112 and to vary the treatment light power and the rate of motion of the treatment light emitter if needed to achieve a uniform or nearly uniform total treatment dose.

FIG. 6 is another illustrative cross-sectional view. The tissue structures, trajectory and control points in FIG. 5 are repeated in FIG. 6. FIG. 6 is an illustrative cross-sectional view of a small portion 150 of a patient comprising a cavity 102 with a targeted region 104 that is highlighted as a black band and is located inside healthy tissue 106. The targeted region is divided into four sub-regions 104a, 104b, 104c and 104d, with the divisions indicated by dashed lines. The number of sub-regions may vary and the number four was chosen for purposes of illustration. The cavity has an opening 108 through which treatment light may enter. An OAR 110 is located near the targeted region 104. The OAR 110 may be sensitive to the total treatment dose and should be subject to significantly less than the threshold value of total treatment dose needed to treat the targeted region 104.

FIG. 6 also includes a light source 22, a treatment light emitter 24 that emits the PDT treatment light, a light transporting device 26 such as an optical fiber device for transporting treatment light from the light source 22 to the treatment light emitter 24 and a multi-axis mechanical or robotic positioning device 28 to control the position of the treatment light emitter 24. Features 22, 24, 26 and 28 are described in the descriptions for FIG. 2 and FIG. 3.

In FIG. 6, an example trajectory 112 for the path of the treatment light emitter 24 is illustrated along with five control points 114. The path of trajectory 112, or optionally multiple trajectories (not shown), and control points 114 are determined and optimized by the treatment planning system 32 (shown in FIG. 2). Light may by emitted only at discrete control points 114 or light may be emitted continuously along the trajectory 112. If light is emitted at discrete control points, for example, the treatment light power and treatment light exposure times may be different for each control point in order to deliver a uniform threshold value of total treatment dose to all or substantially all sub-regions of the entire targeted region 104 while sparing the OAR 110 from a damaging dose.

In FIG. 6, the treatment light emitter 24 is illustrated as located at one of the control points 114 on trajectory 112 and emits light 152 in substantially all directions. To deliver the entire treatment, multi-axis mechanical or robotic positioning device 28 controlled by treatment delivery control system 30 (shown in FIG. 2) moves the treatment light emitter 24 sequentially to each control point 114 along trajectory 112. The treatment delivery control system 30 (shown in FIG. 2) follows a treatment plan that was previously optimized by treatment planning system 32 (shown in FIG. 2).

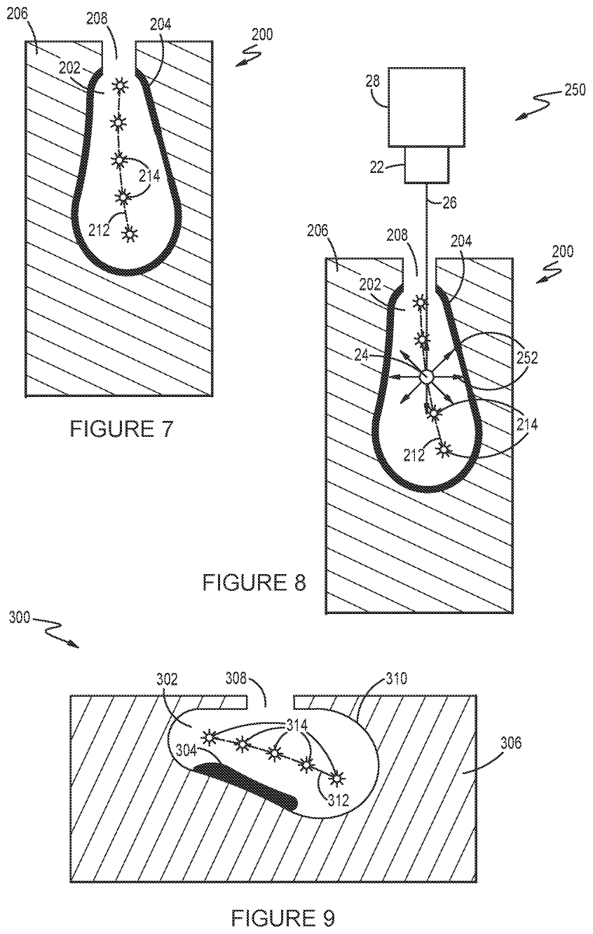

FIG. 7 is another illustrative cross-sectional view of a small portion 200 of a patient comprising a cavity 202 with a targeted region 204 that is highlighted as a black band and is located inside healthy tissue 206. The cavity has an opening 208 through which treatment light may enter. An example trajectory 212 for the path of the treatment light emitter 24 (shown in FIG. 2) is illustrated along with five control points 214. The trajectory 212 and control points 214 are determined and optimized by the treatment planning system 32 (shown in FIG. 2).

In this illustrative example, there are five control points where light is emitted by the treatment light emitter. Light may by emitted only at discrete control points 214 or light may be emitted continuously along the trajectory 212. If light is emitted at discrete control points, for example, the treatment light power and treatment light exposure times may be different for each control point in order to deliver a uniform or nearly uniform threshold value of the total treatment dose to all or substantially all sub-regions of the entire targeted region 204.

FIG. 8 is another illustrative cross-sectional view. The tissue structures, trajectory and control points in FIG. 7 are repeated in FIG. 8. FIG. 8 is an illustrative cross-sectional view of a small portion 250 of a patient comprising a cavity 202 with a targeted region 204 that is highlighted as a black band and is located inside healthy tissue 206. The cavity has an opening 208 through which treatment light may enter. FIG. 8 also includes a light source 22, a treatment light emitter 24 that emits the PDT treatment light, a light transporting device 26 such as optical fiber device or free-space optics for transporting treatment light from the light source 22 to the treatment light emitter 24 and a multi-axis mechanical or robotic positioning device 28 to control the position of the treatment light emitter 24. Features 22, 24, 26 and 28 are described in the descriptions for FIG. 2 and FIG. 3.

In FIG. 8, an example trajectory 212 for the path of the treatment light emitter 24 is illustrated along with five control points 214. The path of trajectory 212, or optionally multiple trajectories (not shown), and control points 214 are determined and optimized by the treatment planning system 32 (shown in FIG. 2). Light may by emitted only at discrete control points 214 or light may be emitted continuously along the trajectory 212. If light is emitted at discrete control points, for example, the treatment light power and treatment light exposure times may be different for each control point in order to deliver a uniform threshold value of the total treatment dose to all or substantially all sub-regions of the entire targeted region 204.

In FIG. 8, the treatment light emitter 24 is illustrated as located at one of the control points 214 on trajectory 212 and emits light 252 in substantially all directions. To deliver the entire treatment, multi-axis mechanical or robotic positioning device 28 controlled by treatment delivery control system 30 (shown in FIG. 2) moves the treatment light emitter 24 sequentially to each control point 214 along trajectory 212. The treatment delivery control system 30 (shown in FIG. 2) follows a treatment plan that was previously optimized by treatment planning system 32 (shown in FIG. 2).

FIG. 9 is another illustrative cross-sectional view of a small portion 300 of a patient comprising a cavity 302 with a targeted region 304 that is highlighted as a black region and is located adjacent to healthy tissue 306. Targeted region 304 covers only a portion of the interior surface 310 of cavity 302. The cavity has an opening 308 through which treatment light may enter. An example trajectory 312 for the path of the treatment light emitter 24 (shown in FIG. 2) is illustrated along with five control points 314. The trajectory 312 and control points 314 are determined and optimized by the treatment planning system 32 (shown in FIG. 2).

In this illustrative example, there are five control points where light is emitted by the treatment light emitter. Light may by emitted only at discrete control points 314 or light may be emitted continuously along the trajectory 312. If light is emitted at discrete control points, for example, the treatment light power and treatment light exposure times may be different for each control point in order to deliver a uniform or nearly uniform threshold value of the total treatment dose to all or substantially all sub-regions of the entire targeted region 304.

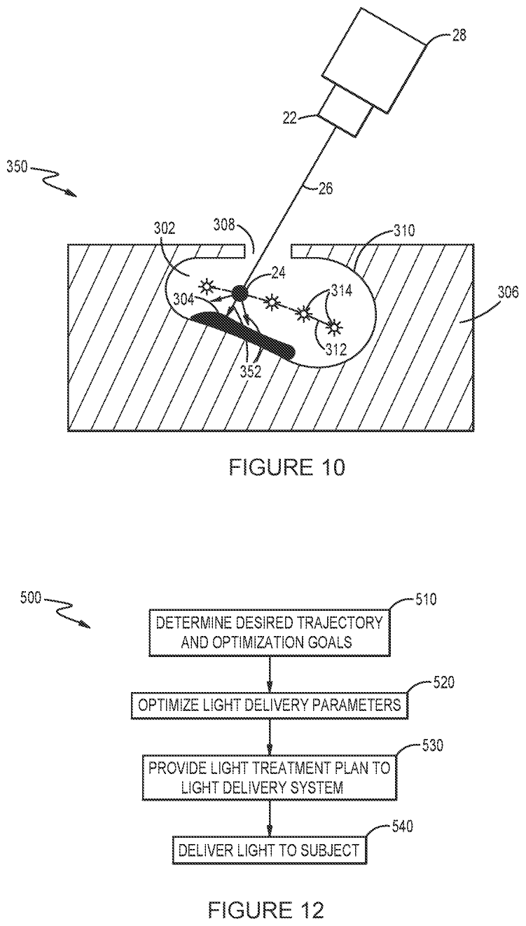

FIG. 10 is another illustrative cross-sectional view. The tissue structures, trajectory and control points in FIG. 9 are repeated in FIG. 10. FIG. 10 is an illustrative cross-sectional view of a small portion 350 of a patient comprising a cavity 302 with a targeted region 304 that is highlighted as a black region and is located adjacent to healthy tissue 306. Targeted region 304 covers only a portion of the interior surface 310 of cavity 302. The cavity has an opening 308 through which treatment light may enter. FIG. 10 also includes a light source 22, a treatment light emitter 24 that emits the PDT treatment light, a light transporting device 26 such as an optical fiber device or free-space optics for transporting treatment light from the light source 22 to the treatment light emitter 24 and a multi-axis mechanical or robotic positioning device 28 to control the position of the treatment light emitter 24. Features 22, 24, 26 and 28 are described in the descriptions for FIG. 2 and FIG. 4.

In FIG. 10, an example trajectory 312 for the path of the treatment light emitter 24 is illustrated along with five control points 314. The path of trajectory 312, or optionally multiple trajectories (not shown), and additional control points 314 are determined and optimized by the treatment planning system 32 (shown in FIG. 2). Light may be emitted only at discrete control points 314 or light may be emitted continuously along the trajectory 312. If light is emitted at discrete control points, for example, the treatment light power and the treatment light exposure time may be different for each control point in order to deliver a uniform or nearly uniform threshold value of the total treatment dose to all or substantially all sub-regions of the entire targeted region 304. In FIG. 10, the treatment light emitter 24 is located at one of the control points 314 on trajectory 312.

In this example, light rays 352 are emitted in a particular direction or directions (for example, the treatment light emitted shape is a cone of light with a fixed or variable angular spread) from the treatment light emitter 24 and are directed preferentially toward the target 304 rather than the entire surface 310 of cavity 302. To deliver the entire treatment, multi-axis positioning device 28 controlled by treatment delivery control system 30 (shown in FIG. 2) moves the treatment light emitter 24 sequentially to each control point 314 along trajectory 312. The treatment delivery control system 30 (shown in FIG. 2) follows a treatment plan that was previously optimized by treatment planning system 32 (shown in FIG. 2).

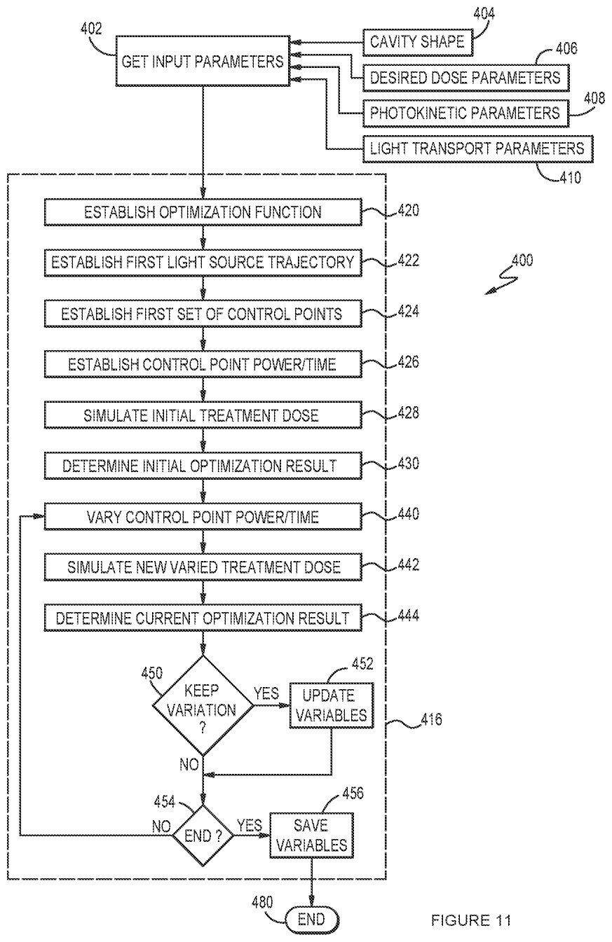

FIG. 11 schematically depicts an example of a method for pretreatment planning 400 for optimizing light dose delivery to a targeted region in accordance with the present disclosure. In some embodiments, method 400 provides a pretreatment plan that is designed to deliver at least a threshold total treatment dose to all or substantially all sub-regions of the entire targeted region and that is also within an acceptable tolerance so as to avoid significantly damage healthy tissue and organs-at-risk. The delivery of the appropriate treatment dose may be achieved by mechanically or robotically moving the treatment light emitter along a trajectory while varying the treatment light power, and treatment light exposure time in some embodiments. Optionally or alternatively, the treatment light emitted shape at each control point on the trajectory may be adjusted. In some embodiments, the appropriate treatment dose may be achieved, by further varying the treatment light power, treatment light exposure time and/or the treatment light emitted shape continuously along the trajectory.

Method 400 may be performed, at least in part, by a treatment planning system 32 illustrated in FIG. 2. In some embodiments, the method 400 comprises getting input parameters, estimating an optimization function, estimating a first trajectory, estimating first control points, and estimating an initial set of treatment light parameters at each control point. The initial set of treatment light parameters may include treatment light power, the initial treatment light exposure time and, optionally, the initial treatment light emitted shape. In addition, the method 400 further comprises simulating an initial total treatment dose, determining an initial result, and then, if necessary, iterating one or more times by varying one or more of the set of treatment light parameters at each control point.

The following explanation of method for pretreatment planning 400 in FIG. 11 utilizes the diagram in FIG. 6 as an illustrative example. FIG. 6 comprises cavity 102, targeted region 104 that substantially covers the surface of cavity 102, sub-regions 104a, 104b, 104c and 104d, healthy tissue 106, organ-at-risk 110, treatment light emitter 24, trajectory 112 and control points 114.

In method 400, at 402 input parameters for the treatment plan are obtained. The input parameters may comprise the cavity shape 404, the desired dose parameters 406, photokinetic parameters 408 and light transport parameters 410. The cavity shape 404 may be obtained using imaging techniques such as, for example, computed tomography (CT), positron emission tomography (PET), magnetic resonance imaging (MRI), ultrasound imaging or 3D optical imaging. The desired dose parameters 406 are preferably the threshold total treatment dose parameters effective for the treatment. Preferably, a total treatment dose is greater than a threshold light dose, a threshold PDT-dose, a threshold reactive oxygen species dose and/or a threshold reactive singlet oxygen dose. The total treatment dose, in some embodiments, may be determined as a sum of treatment dose resulting from the light emitted by the treatment light emitter from control points that result in a dose rate greater than a threshold dose rate. The total treatment threshold dose may be different for different target types and different photosensitizers. The photokinetic parameters 408 may be different for different photosensitizers. The light transport parameters 410 comprise the light absorption parameter, mua (or μₐ) the light scattering parameter mus (or μₛ), the scattering anisotropy factor, gₛ, and the index of refraction, n. Note that the scattering anisotropy factor, gₛ, is a different parameter than the photokinetic parameter, g, for the oxygen intake rate. The light transport parameters 410 may be different for each material (e.g. targeted region, healthy tissue, air) in the simulation.

After the input parameters are obtained, method 400 then proceeds to an optimization process, at 416, that determines the effective treatment light parameters such as, for example, the treatment light power, treatment light exposure time and/or treatment light emitted shape for each control point 114 of trajectory 112. During the optimization process 416, the treatment light powers, treatment light exposure times and, optionally, treatment light emitted shapes are iteratively varied. Variations are accepted if the results are improved or rejected if the results are worse than the previous iteration. Optimization process 416 continues until it either achieves an acceptable result or fails to converge to an acceptable result.

The optimization process further includes, at 420, estimating an optimization function that determines the goodness of the result. An example optimization function is a least-squares optimization function S that is shown in Equation (14), but other types of optimization functions may be utilized.

S=Σᵢ^{sub-region}((Σⱼ^{Control Points}[Calculated dose]_{i,j})−[Desired dose])² (14)

In this example, the function S is minimized to obtain the effective set of parameters. The targeted region is assumed to be divided into smaller sub-regions, i.e., sub-areas or sub-volumes, since the dose rate and treatment dose may be different for different portions of the targeted region. FIG. 6 illustrates an example including sub-regions 104a, 104b, 104c and 104d. For each sub-region i (a portion of the total treatment area or total treatment volume, respectively) on the targeted region of the cavity, the square of the difference between the calculated treatment dose and the desired threshold treatment dose is determined. Such difference for each cavity surface sub-region i is then summed for all sub-regions to get S. The total treatment dose to a sub-region i is a sum of the incremental treatment doses calculated for all the control points j-s, [Calculated dose]_{i,j}, delivered to sub-region i while the treatment light emitter is at control point j. Preferably, the incremental treatment dose [Calculated dose]_{i,j} is added to the total treatment dose for each sub-region i and for each control point j only when the dose rate to the sub-region is greater than a threshold dose rate when the light source is at control point j. If a minimum threshold dose rate is desired during optimization for a sub-region i, then in Equation (14), the [Calculated dose]_{i,j} is set to 0 (zero) if [Dose rate]_{i,j}<[Threshold dose rate]. Preferably, at least 75% of the targeted region receives threshold treatment dose during the time when at least threshold dose rate is applied to each sub-region. More preferably, at least 90% of the targeted region receives threshold treatment dose during the time when at least threshold dose rate is applied to each sub-region. Most preferably, at least 99% of the targeted region receives threshold treatment dose during the time when at least threshold dose rate is applied to each sub-region. However, it will be appreciated that the determination of which (or how much of) portion of the targeted region receives at least the threshold dose may be determined by the clinician based on the clinician's judgement. Thus, a smaller portion than 75% of the targeted region may also be selected for receiving the threshold treatment dose in some embodiments.

At 422, a first trajectory 112 for the treatment light emitter 24 is estimated. The trajectory 112 may be straight or curved. The trajectory may be determined manually or by computer calculation.

At 424, first control points along the trajectory are estimated. If the cavity 102 is spherical in shape, one control point 114 positioned at the center of the cavity may be sufficient. In general, however, the cavity shape is not spherical and two or more control points may be estimated for optimization for achieving an acceptable uniform treatment result. The locations of the control points may be determined manually or by computer calculation.

At 426 initial treatment light parameters are estimated for each control point 114 (shown in FIG. 6). The initial treatment light parameters may include, but are not limited to treatment light power, treatment light exposure time and/or treatment light emitted shape. The multi-axis mechanical or robotic positioning system sequentially positions the treatment light emitter at each of the first control points by controlling one or more axis positions of the treatment light emitter relative to the interior surface of the cavity of the patient. The initial treatment light parameters may be the same for all control points 114 or may be different for each control points. The treatment light emitted shape may be, for example, substantially uniform or isotropic in all directions or the treatment light emitted shape may direct light preferentially in one or more directions. The initial treatment light power may be the same for all control points or may be different for each control point. The initial treatment light exposure time may be the same for all control points or may be different for each control point.

At 428, the initial treatment dose for each sub-region i of the cavity surface and for each control point j is simulated.

At 430, the optimization function to calculate the initial optimization result is utilized. In some embodiments, the optimization function may be the solution S in Equation (14)).

At 440, one or more of the treatment light parameters for each control point j are varied. For example, one or more of treatment light power, the treatment light exposure time and/or the treatment light emitted shape are varied for each control point j.

At 442, the changed treatment light parameters are used to simulate a changed treatment dose for each sub-region i of the cavity surface and for each control point j.

At 444, the optimization function is used to calculate a current optimization result (for example, calculating the new value for S in Equation (14)).

At 450, it is determined whether the current optimization result (e.g., variation S) determined at 444, is better (smaller) or worse (larger) than the initial optimization result. If the variation is better (YES), the variables are updated, at 452, and the process moves to 454. If the variation is worse (NO), the process moves directly to 454.

At 454, a decision is made whether to end the optimization (YES) or to return to 440 (NO) to try a new variation of one or more of the treatment light parameters such as power, the treatment light exposure time and, optionally, the treatment light emitted shape. If the decision at 450 was YES and the variables were updated at 452, then decision at 454 is NO and the optimization is continued. If the decision at 450 was NO, then the decision at 454 is YES and final values for the variables are saved at 456 and the optimization ended at 480.

Iteration at 440 through 454 in FIG. 11 continues until a decision is made to stop the optimization process at 480.

Method 400 illustrated in FIG. 11 is part of an overall method for planning and delivering light treatment to a patient.

FIG. 12 schematically illustrates an example of a method 500 for planning and delivery of treatment light to a patient in accordance with the present disclosure. The following explanation of method 500 in FIG. 12 utilizes the diagrams in FIG. 6 and FIG. 2 as illustrative examples.

At 510 a desired trajectory 112 and desired optimization goals are determined. Desired optimization goals may be, for example, making sure that each sub-region of the targeted region 104 on the surface of cavity 102 receives the desired total treatment dose. Method 500 then moves to 520 where a set of light delivery parameters are optimized. In some embodiments, 520 may comprise optimization of one or more of the treatment light power, the treatment light exposure time and/or the treatment light emitted shape for each control point 114 of trajectory 112 utilizing method 400. The result of optimization at 520 is a light treatment plan. The light treatment plan, at 530, is provided to the treatment delivery control system 30 (illustrated in FIG. 2). The light treatment delivery system, at 540, then delivers the treatment light to the targeted region of the patient 42 (illustrated in FIG. 2) according to the light treatment plan developed at 520.

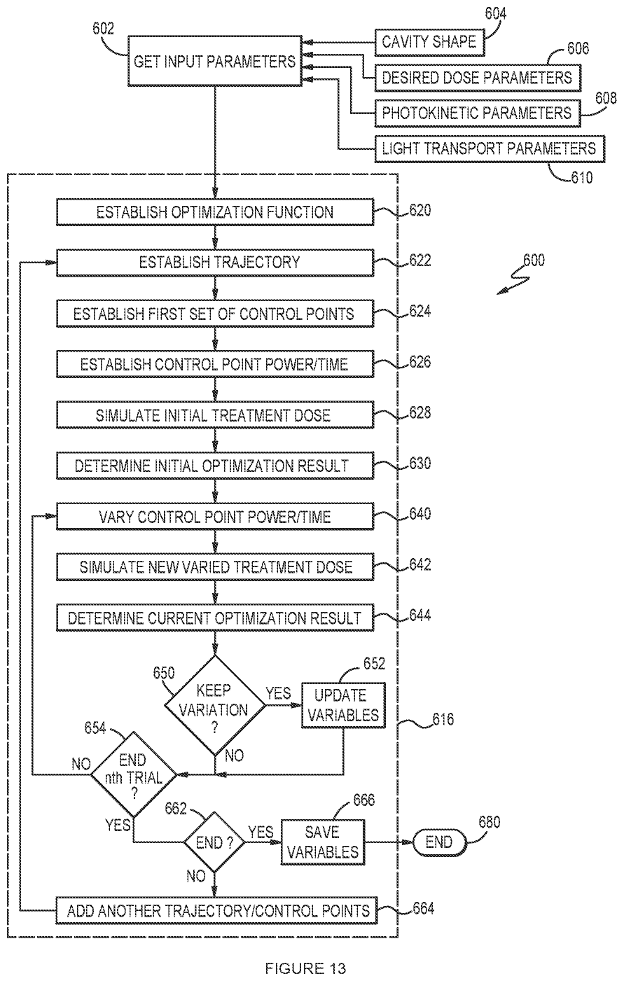

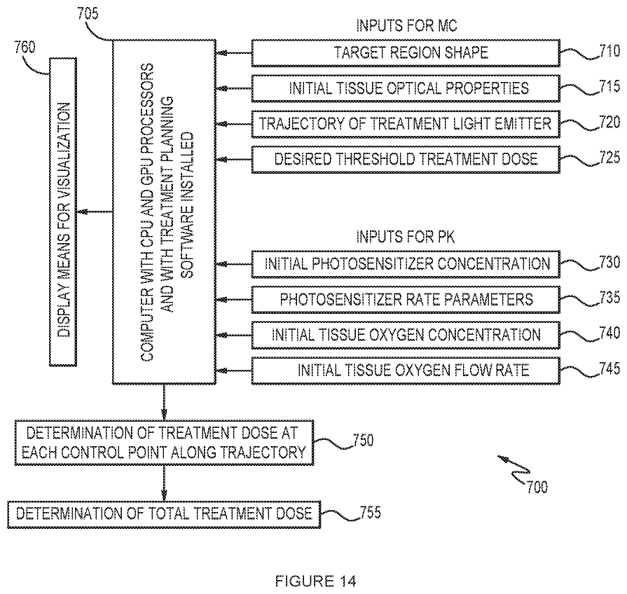

FIG. 13 shows another example of a method for pretreatment planning 600 for optimizing light dose delivery to a targeted region in accordance with the present disclosure. The method 600 is designed to provide a light treatment plan that delivers at least a threshold total treatment dose to all or substantially all sub-regions of the entire targeted region and that is also within an acceptable tolerance that avoids significant damage to healthy tissue and organs-at-risk. This can be achieved by moving the treatment light emitter along a first trajectory and at least a second trajectory while varying one or more of a set of treatment light parameters at each of a first set of control points on the first trajectory and at each of at least a second set of control points on at least a second trajectory. The set of treatment light parameters include, but are not limited to, the treatment light power, the treatment light exposure time and/or the treatment light emitted shape. Optionally or alternatively, varying the set of treatment light parameters may be varied continuously along the first trajectory and at least a second trajectory. The first and second trajectories may be determined manually or by computer calculation. The multi-axis mechanical or robotic positioning system is used to sequentially position the treatment light emitter at each of the first set of control points and the at least second set of control points by controlling one or more axis positions of the treatment light source relative to the patient.

Method 600 of FIG. 13 may be used as a part of 520 of method 500 in FIG. 12 in some embodiments. Method 600 of FIG. 13 may be similar to method 400 of FIG. 11 in some embodiments. Method 600 comprises a number of processes, e.g., those at 602 through 654 that are substantially equivalent to those at 402 through 454 of method 400. In method 400 of FIG. 11, the optimization uses a first trajectory established at 422 and a first set of control points established at 424. The optimization in method 400 then proceeds to 454 where a decision is made either to end the optimization and proceed to 456 or to try another iteration of varying the set of treatment light parameters at each control point by returning to 440.

In method 600 of FIG. 13, the optimization using a first trajectory and a first set of control points proceeds to 654 to decide whether to end an nth trial for the first trajectory and the first set of control points (YES) or try another iteration (NO) by adding another trajectory and to vary, for each control point of the new trajectory, the set of treatment light parameters.

The following explanation of method for pretreatment planning 600 in FIG. 13 utilizes the diagram in FIG. 6 as an illustrative example. FIG. 6 comprises cavity 102, targeted region 104 that substantially covers the surface of cavity 102, healthy tissue 106, organ-at-risk 110, treatment light emitter 24, trajectory 112 and control points 114.

In method 600, at 602 input parameters for the treatment plan are obtained. The input parameters may comprise the cavity shape 604, the desired dose parameters 606, photokinetic parameters 608 and light transport parameters 610.

The cavity shape may be obtained using imaging techniques such as, for example, computed tomography (CT), positron emission tomography (PET), magnetic resonance imaging (MRI), ultrasound imaging or 3D optical imaging.

The total treatment dose parameter 606 is preferably the threshold total treatment dose to all or substantially all sub-regions of the targeted region selected for the treatment. Preferably the total treatment dose is greater than a threshold light dose, a threshold PDT-dose, a threshold reactive oxygen species dose or a threshold reactive singlet oxygen dose. The total treatment threshold dose may be different for different target types and different photosensitizers.

The photokinetic parameters 608 may be different for different photosensitizers.

The light transport parameters 610 comprise the light absorption parameter, mua (or μₐ) the light scattering parameter mus (or μₛ), the scattering anisotropy factor, gₛ, and the index of refraction, n. Note that the scattering anisotropy factor, gₛ, is a different parameter than the photokinetic parameter, g, for the oxygen intake rate. The light transport parameters 610 may be different for each material (e.g. targeted region, healthy tissue, air) in the simulation.

After the input parameters are obtained, method 600 then proceeds to an optimization process 616 that determines effective treatment light parameters, which include, but are not limited to, light treatment powers, treatment light exposure times and/or treatment light emitted shapes, for each control point 114 of trajectory 112. During the optimization process 616, the treatment light parameters may be iteratively varied. Variations are accepted if the results are improved or rejected if the results are worse than the previous iteration. Optimization process 616 is continued until it either an acceptable result is achieved or has failed to converge to an acceptable result.

The optimization process includes, at 620, estimating an optimization function for determining the goodness of the result. An example optimization function is a least-squares optimization function S that is shown in Equation (14), but other types of optimization functions may be utilized. In this example, the function S is minimized to get the effective result. The targeted area is assumed to be divided into smaller sub-regions. For each sub-region i (a portion of the total treatment area or volume) on the targeted region of the cavity and for each control point j, the difference between the calculated treatment dose and the desired threshold treatment dose is calculated. Next the square of the difference for each cavity surface sub-region i and for each control point j position of the treatment light emitter is calculated and the results are summed to obtain S as shown in Equation (14).

At 622, a first trajectory 112 for the treatment light emitter 24 is estimated. The trajectory 112 may be straight or curved. The trajectory may be determined manually or by computer calculation.

At 624 first control points along the trajectory are estimated. If the cavity 102 is spherical in shape, one control point 114 positioned at the center of the cavity may be sufficient. In general, the cavity shape is not spherical and two or more control may be used for optimization for achieving an acceptable uniform treatment result. The control points may be determined manually or by computer calculation.

At 626, initial treatment light parameters are estimated for each control point 114 of FIG. 6. The treatment light parameters may include, without limitation, the treatment light power, treatment light exposure time and, optionally, treatment light emitted shape. The multi-axis mechanical or robotic positioning system may sequentially position the treatment light emitter at each of the first control points by controlling one or more axis positions of the treatment light emitter relative to the interior surface of the cavity of the patient. The initial treatment light parameters may be the same for all control points 114 or may be different for each control points. The treatment light emitted shape may be, for example, substantially uniform or isotropic in all directions or the treatment light emitted shape may direct light preferentially in one or more directions. The initial treatment light power may be the same for all control points or may be different for each control point. The initial treatment light exposure time may be the same for all control points or may be different for each control point.

At 628, the initial treatment dose for each sub-region i of the cavity surface and for each control point j is simulated.

At 630 the initial optimization result (for example, using solution S in Equation (14)) is calculated using the optimization function.

At 640, one or more of the set of treatment light parameters are varied for each control point j to obtain a changed set of (new) treatment light parameters.

At 642, a changed (new) treatment dose for each sub-region i of the cavity surface and for each control point j is simulated using the changed set of treatment light parameters obtained at 640.

At 644, a current (new) optimization result (for example, using the new value for S in Equation (14)) is calculated using the optimization function.

At 650, it is determined whether the current (new) variation S is better (smaller) or worse (larger) than the initial result for S. If the variation is better (YES), the variables are updated at 652 and the process moves to 654. If the variation is worse (NO), the process moves directly to 654.

At 654, a decision is made whether to end the optimization (YES) or to return to 640 (NO) to try a new variation of the set of treatment light parameters. If the decision at 650 was YES and the variables were updated at 652, then decision at 654 should be NO and the optimization should continue with the current trajectory. If the decision at 650 was NO, then the decision at 654 should be YES and the process moves to 662 to add another trajectory.

If the optimization using the first trajectory and the first set of control points is terminated, method 600 includes a decision, at 662, to determine if another trajectory and another set of control points are to be added to the treatment plan or whether to end the optimization. Initially, the decision at 662 should be NO and another trajectory and another set of control points are added at 664 to the optimization plan. The optimization returns to 622 and an added total treatment dose is simulated using the new trajectory and new set of control points. If the new trajectory and control points do not improve the solution, then the decision at 662 is YES and the latest variables are saved at 666 and the method proceeds to the END at 680.

A preferred treatment planning system 700 shown in FIG. 14 (also shown as 32 in FIG. 2) for generating an intracavitary treatment plan comprises a computer 705 with one or more CPUs (central processing units), one or more GPUs (graphical processing units), and memory, preferably non-transitory memory, having computer program instructions (e.g., a software) stored thereon for implementing any of the methods described herein. In an example, the computer program instructions may be a proprietary software such as Dosie™ provided by Simphotek, Inc.

The software, implementing any of the methods described herein, can calculate the light dose, light fluence rate, PDT-dose, [ROS]_{dose} and [¹O₂]_{dose} for each of a plurality of computational spatial elements for intracavitary photodynamic therapy and for each control point along a trajectory for the treatment light emitter. In some embodiments, the software may combine, in one integrated device, Monte Carlo (MC) and finite element (FE) simulations of light transport, light dose (fluence), light fluence rate and as well as photokinetics (PK) simulations needed for calculations of [ROS]_{dose}, [¹O₂]_{dose}, and PDT-dose. The cavity computational spatial elements used for the simulations may be cubical voxels or tetrahedrons for MC simulations and tetrahedrons for FE simulations. Preferably, MC techniques with cubical voxels are used for intracavitary PDT simulations. The light fluence rate simulations for each of the plurality of cavity computational spatial elements in a cavity (for example, cavity 40 in FIG. 2) may use as MC inputs the target region shape 710, the initial tissue optical properties 715 of the cavity walls, the trajectory 720 of the treatment light emitter and the desired threshold treatment dose 725.

The trajectory 720 can be determined by calculations performed, e.g., by a computer 705, or by manual inputs from a user of the computer. In some embodiments, computer 705 may calculate the treatment dose 750 for each sub-region at each control point along the trajectory by determining the best treatment light emitter power and the corresponding treatment time at each control point. The treatment doses 750 for each sub-region for each of the control points to determine the total treatment dose 755 are then summed. The PK simulations may, in some embodiments, use the light fluence rate results, the photokinetic Equations (1)-(13), plus, if known, inputs of initial PS concentration 730, PS rate parameters 735, initial tissue oxygen concentration 740 and oxygen flow rates 745 to calculate [ROS]_{dose}, [¹O₂]_{dose}, and PDT-dose. If the initial PS concentration 730, PS rate parameters 735, initial tissue oxygen concentration 740 and oxygen flow rates 745 are not known, they can be approximated using values obtained from published literature. The calculated PDT photokinetics include light-PS-excitation, the PS-to-oxygen excitation to generate singlet oxygen, the singlet oxygen reaction with the target region and the singlet oxygen reaction with the PS (resulting in photobleaching).

In some embodiments, the computer 705 may include a display to provide graphics 760 relating to treatment planning system 700. The graphics 760 may display 2D and 3D outputs of light fluence (light dose), light fluence rate, PDT-dose, [ROS]_{dose} and [¹O₂]_{dose} at every computational spatial element in the cavity and cavity walls. A clinician can use this information to localize areas of under-treatment and make corrections, if needed, to the treatment plan.

Thus, the systems and method disclosed herein provide for improved pretreatment planning and treatment delivery for intracavitary photodynamic therapy such that at least a threshold treatment dose is provided to the affected regions in the cavity while avoiding damage to healthy tissue and organs-at-risk.

While several exemplary aspects and embodiments have been discussed above, those having skill in the art will recognize certain modifications, permutations, additions and sub-combinations that are also within the spirit and scope of this invention.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1: A method of delivering treatment light for intracavitary photodynamic therapy to a targeted region within a cavity of a patient, the method comprising:
generating a treatment plan for delivering the treatment light to generate a total treatment dose to the targeted region by:
   receiving, at a processor, shape information for an interior surface of the cavity;
   initializing, by the processor, a first set of control points located on a first trajectory within the cavity by assigning, to each of the first set of control points, one or more axis positions of a treatment light emitter relative to the interior surface of the cavity, the first trajectory defining a relative motion between a treatment light emitter and the interior surface of the cavity;
   iteratively optimizing, by the processor, a simulated total treatment dose relative to a set of one or more optimization goals when the treatment light emitter is activated to emit treatment light at each of the first set of control points; and
   determining, by the processor, a treatment plan by assigning values for a set of treatment light delivery parameters to each of the first set of control points; and
delivering the treatment light to the targeted region within the cavity by activating the treatment light emitter in accordance with the treatment plan.

Clause 2: The method of any of the preceding clauses, wherein the set of treatment light delivery parameters include treatment light power and treatment light exposure time.

Clause 3: The method of clause 2, wherein the set of treatment light delivery parameters further includes treatment light emitted shape.

Clause 4: The method of any of the preceding clauses, wherein the targeted region comprises a plurality of sub-regions, wherein the total treatment dose to each sub-region within the cavity is a sum of each incremental treatment dose to sub-regions generated when the treatment light emitter is consecutively activated to emit light at each of the first set of control points and when an applied dose rate from the treatment light emitter to a corresponding sub-region is greater than a threshold dose rate.

Clause 5: The method of clause 4, wherein optimizing the simulated total treatment dose to the targeted region comprises determining the total treatment dose at each sub-region of the targeted region that is greater than at least a threshold treatment dose, provided that at least a threshold dose rate is applied to sub-regions from the treatment light emitted from the first set of control points.

Clause 6: The method of one of clause 4 or 5, wherein optimizing the simulated total treatment dose further comprises determining the total treatment dose at each of the first set of control points that is lower than a threshold treatment dose at regions within the cavity other than the targeted region and at organs-at-risk outside the cavity near the targeted region.

Clause 7: The method of any of the preceding clauses, wherein treatment dose comprises one or more of a light dose, a PDT-dose, a reactive oxygen species dose and a reactive singlet oxygen dose.

Clause 8: The method of any of the preceding clauses, wherein generating the plan further comprises initializing at least a second set of control points along at least a second trajectory within the cavity and iteratively optimizing a simulated second total treatment dose relative to a second set of one or more optimization goals over the second set of control points to determine a second set of treatment light delivery parameters corresponding to each of the second set of control points.

Clause 9: A system for delivery of treatment light for intracavitary photodynamic therapy to a targeted region within a cavity of a patient, the system comprising:
a treatment light emitter;
a positioning device configured to move the treatment light emitter relative to a first set of control points located on a first trajectory within the cavity;
a controller configured to receive a treatment plan and, in accordance with the treatment plan:
   cause the positioning device to effect relative movement between the treatment light emitter and an interior surface of the cavity to enable the treatment light emitter to arrive at each of the first set of control points along the first trajectory;
   while at each of the first set of control points, cause the treatment light emitter to be activated to emit light; and
   cause values of a set of treatment light delivery parameters of the treatment light emitter to vary in accordance with the treatment plan while the treatment light emitter is moved through the first set of control points along the first trajectory.

Clause 10: The system of clause 9 further comprising: a treatment planning subsystem configured to generate the treatment plan for delivering the treatment light to generate a total treatment dose to the targeted region that is greater than a threshold total treatment dose.

Clause 11: The system of clause 10, wherein the treatment planning subsystem is configured to generate the treatment plan by:
receiving, at a processor, shape information for an interior surface of the cavity;
initializing, by the processor, a first set of control points located on a first trajectory within the cavity by assigning, to each of the first set of control points, one or more axis positions of a treatment light emitter relative to the interior surface of the cavity, the first trajectory defining a relative motion between the treatment light emitter and the interior surface of the cavity;
iteratively optimizing, by the processor, a simulated total treatment dose relative to a set of one or more optimization goals when the treatment light emitter is activated to emit treatment light at each of the first set of control points; and
determining, by the processor, a treatment plan by assigning values for a set of treatment light delivery parameters to each of the first set of control points.

Clause 12: The system of clause 11, wherein the set of treatment light delivery parameters include treatment light power and treatment light exposure time.

Clause 13: The system of clause 12, wherein the set of treatment light delivery parameters further includes treatment light emitted shape.

Clause 14: The system of any one of clauses 11-13, wherein the targeted region comprises a plurality of sub-regions, wherein the total treatment dose to each sub-region within the cavity is a sum of each incremental treatment dose to sub-regions generated when the treatment light emitter is consecutively activated to emit light at each of the first set of control points and when an applied dose rate from the treatment light emitter to a corresponding sub-region is greater than a threshold dose rate.

Clause 15: The system of clause 14, wherein optimizing the simulated total treatment dose to the targeted region comprises determining the total treatment dose at each sub-region of the targeted region that is greater than at least a threshold treatment dose, provided that at least a threshold dose rate is applied to sub-regions from the treatment light emitted from the first set of control points.

Clause 16: The system of clause 14, wherein optimizing the simulated total treatment dose further comprises determining total treatment dose at each of the first set of control points that is lower than the threshold treatment dose at regions within the cavity other than the targeted region and at organs-at-risk outside the cavity near the targeted region.

Clause 17: The system of any one of clauses 11-16, wherein treatment dose comprises one or more of a light dose, a PDT-dose, a reactive oxygen species dose and a reactive singlet oxygen dose.

Clause 18: The system of any one of clauses 11-18, wherein generating the plan further comprises initializing at least a second set of control points along at least a second trajectory within the cavity and iteratively optimizing a simulated second total treatment dose relative to a second set of one or more optimization goals over the second set of control points to determine a second set of treatment light delivery parameters corresponding to each of the second set of control points.

Clause 19: The system of any one of clauses 11-18, further comprising: a display device configured to display the received treatment plan to a user of the system.

Clause 20: A non-transitory computer-readable medium comprising instructions, which when executed by a processor, cause the processor to perform operations comprising:
receiving, at the processor, shape information for an interior surface of a cavity;
initializing, by the processor, a first set of control points located on a first trajectory within the cavity by assigning, to each of the first set of control points, one or more axis positions of a treatment light emitter relative to the interior surface of the cavity, the first trajectory defining a relative motion between a treatment light emitter and the interior surface of the cavity;
iteratively optimizing, by the processor, a simulated total treatment dose relative to a set of one or more optimization goals when the treatment light emitter is activated to emit treatment light at each of the first set of control points; and
determining, by the processor, a treatment plan by assigning values for a set of treatment light delivery parameters to each of the first set of control points.

Clause 21: The method of clause 4 or the system of clause 14, wherein the total treatment dose is greater than a threshold dose for at least 75% of the sub-regions of the targeted region.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the term “about” preceding a quantity indicates a variance from the quantity. The variance may be caused by manufacturing tolerances or may be based on differences in measurement techniques. The variance may be up to 10% from the listed value in some instances. Those of ordinary skill in the art would appreciate that the variance in a particular quantity may be context dependent and thus, for example, the variance in a dimension at a micro or a nano scale may be different than variance at a meter scale.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A method of delivering treatment light for intracavitary photodynamic therapy to a targeted region within a cavity of a patient, the method comprising:
generating a treatment plan for delivering the treatment light to generate a total treatment dose to the targeted region by:
receiving, at a processor, shape information for an interior surface of the cavity;
initializing, by the processor, a first set of control points located on a first trajectory within the cavity by assigning, to each of the first set of control points, one or more axis positions of a treatment light emitter relative to the interior surface of the cavity, the first trajectory defining a relative motion between a treatment light emitter and the interior surface of the cavity;
iteratively optimizing, by the processor, a simulated total treatment dose relative to a set of one or more optimization goals when the treatment light emitter is activated to emit treatment light at each of the first set of control points; and
determining, by the processor, a treatment plan by assigning values for a set of treatment light delivery parameters to each of the first set of control points; and
delivering the treatment light to the targeted region within the cavity by activating the treatment light emitter in accordance with the treatment plan.

2. The method of claim 1, wherein the set of treatment light delivery parameters include treatment light power and treatment light exposure time.

3. The method of claim 2, wherein the set of treatment light delivery parameters further includes treatment light emitted shape.

4. The method of claim 1, wherein the targeted region comprises a plurality of sub-regions, wherein the total treatment dose to each sub-region within the cavity is a sum of each incremental treatment dose to sub-regions generated when the treatment light emitter is consecutively activated to emit light at each of the first set of control points and when an applied dose rate from the treatment light emitter to a corresponding sub-region is greater than a threshold dose rate.

5. The method of claim 4, wherein optimizing the simulated total treatment dose to the targeted region comprises determining the total treatment dose at each sub-region of the targeted region that is greater than at least a threshold treatment dose, provided that at least a threshold dose rate is applied to sub-regions from the treatment light emitted from the first set of control points.

6. The method of claim 4, wherein optimizing the simulated total treatment dose further comprises determining the total treatment dose at each of the first set of control points that is lower than a threshold treatment dose at regions within the cavity other than the targeted region and at organs-at-risk outside the cavity near the targeted region.

7. The method of claim 1, wherein treatment dose comprises one or more of a light dose, a PDT-dose, a reactive oxygen species dose and a reactive singlet oxygen dose.

8. The method of claim 1, wherein generating the plan further comprises initializing at least a second set of control points along at least a second trajectory within the cavity and iteratively optimizing a simulated second total treatment dose relative to a second set of one or more optimization goals over the second set of control points to determine a second set of treatment light delivery parameters corresponding to each of the second set of control points.

9. A system for delivery of treatment light for intracavitary photodynamic therapy to a targeted region within a cavity of a patient, the system comprising:
a treatment light emitter;
a positioning device configured to move the treatment light emitter relative to a first set of control points located on a first trajectory within the cavity;
a controller configured to receive a treatment plan and, in accordance with the treatment plan:
cause the positioning device to effect relative movement between the treatment light emitter and an interior surface of the cavity to enable the treatment light emitter to arrive at each of the first set of control points along the first trajectory;
while at each of the first set of control points, cause the treatment light emitter to be activated to emit light; and
cause values of a set of treatment light delivery parameters of the treatment light emitter to vary in accordance with the treatment plan while the treatment light emitter is moved through the first set of control points along the first trajectory.

10. The system of claim 9 further comprising:
a treatment planning subsystem configured to generate the treatment plan for delivering the treatment light to generate a total treatment dose to the targeted region that is greater than a threshold total treatment dose.

11. The system of claim 10, wherein the treatment planning subsystem is configured to generate the treatment plan by:
receiving, at a processor, shape information for an interior surface of the cavity;
initializing, by the processor, a first set of control points located on a first trajectory within the cavity by assigning, to each of the first set of control points, one or more axis positions of a treatment light emitter relative to the interior surface of the cavity, the first trajectory defining a relative motion between the treatment light emitter and the interior surface of the cavity;
iteratively optimizing, by the processor, a simulated total treatment dose relative to a set of one or more optimization goals when the treatment light emitter is activated to emit treatment light at each of the first set of control points; and
determining, by the processor, a treatment plan by assigning values for a set of treatment light delivery parameters to each of the first set of control points.

12. The system of claim 11, wherein the set of treatment light delivery parameters include treatment light power and treatment light exposure time.

13. The system of claim 12, wherein the set of treatment light delivery parameters further includes treatment light emitted shape.

14. The system of claim 11, wherein the targeted region comprises a plurality of sub-regions, wherein the total treatment dose to each sub-region within the cavity is a sum of each incremental treatment dose to sub-regions generated when the treatment light emitter is consecutively activated to emit light at each of the first set of control points and when an applied dose rate from the treatment light emitter to a corresponding sub-region is greater than a threshold dose rate.

15. The system of claim 14, wherein optimizing the simulated total treatment dose comprises determining the total treatment dose at each sub-region of the targeted region that is greater than at least a threshold treatment dose, provided that at least a threshold dose rate is applied to sub-regions from the treatment light emitted from the first set of control points.

16. The system of claim 14, wherein optimizing the simulated total treatment dose further comprises determining the total treatment dose at each of the first set of control points that is lower than the threshold treatment dose at regions within the cavity other than the targeted region and at organs-at-risk outside the cavity near the targeted region.

17. The system of claim 11, wherein treatment dose comprises one or more of a light dose, a PDT-dose, a reactive oxygen species dose and a reactive singlet oxygen dose.

18. The system of claim 11, wherein generating the plan further comprises initializing at least a second set of control points along at least a second trajectory within the cavity and iteratively optimizing a simulated second total treatment dose relative to a second set of one or more optimization goals over the second set of control points to determine a second set of treatment light delivery parameters corresponding to each of the second set of control points.

19. The system of claim 9 further comprising:
a display device configured to display the received treatment plan to a user of the system.

20. A non-transitory computer-readable medium comprising instructions, which when executed by a processor, cause the processor to perform operations comprising:
receiving, at the processor, shape information for an interior surface of a cavity;
initializing, by the processor, a first set of control points located on a first trajectory within the cavity by assigning, to each of the first set of control points, one or more axis positions of a treatment light emitter relative to the interior surface of the cavity, the first trajectory defining a relative motion between a treatment light emitter and the interior surface of the cavity;
iteratively optimizing, by the processor, a simulated total treatment dose relative to a set of one or more optimization goals when the treatment light emitter is activated to emit treatment light at each of the first set of control points; and
determining, by the processor, a treatment plan by assigning values for a set of treatment light delivery parameters to each of the first set of control points.
